(19) United States
(12) Patent Application Publication
(10) Pub. No.: US 2014/0170196 A1
(43) Pub. Date: Jun. 19, 2014

(54) FEMININE ANTI-ITCH CLOTH

(71) Applicants: Bobby Corry, Nathalie, VA (US); Russ Hovda, Lynchburg, VA (US); Michael L. Caswell, Lynchburg, VA (US)

(72) Inventors: Bobby Corry, Nathalie, VA (US); Russ Hovda, Lynchburg, VA (US); Michael L. Caswell, Lynchburg, VA (US)

(21) Appl. No.: 13/720,208

(22) Filed: Dec. 19, 2012

(51) Int. Cl. A61K 9/00

(52) U.S. Cl. CPC A61K 9/0034
USPC 424/402; 514/239.2; 427/2.31

(57) ABSTRACT

The present invention provides disposable cloths that are designed to reduce itching discomfort when wiped across the vulvar tissue. These disposable cloths are wetted with a composition that includes at least one anesthetic, protectant, buffer, and surfactant. The composition is substantially clear and preferably does not bring about a cooling or stinging sensation when applied to the vulvar tissue. Furthermore, the composition has a pH adapted to vulvar use.

### REFERENCE TO RELATED APPLICATIONS

This application claims the benefit of co-pending U.S. application Ser. No. 11/027,492 entitled “Feminine Anti-Itch Cloth” filed Dec. 30, 2004, which is incorporated by reference in its entirety.

### BACKGROUND

Itching and the associated discomfort that accompanies skin irritation are common annoyances. The discomfort caused by itching is especially problematic when experienced at the rectal or vulvar tissues of the body. Vulvar tissues form the surfaces of the female genitalia, but do not extend into the vaginal canal.

One effective method of reducing the discomfort from rectal itching is through the use of anorectal pads, such as FLEET® Pain-Relief Pre-Moistened Anorectal Pads available from C.B. FLEET Company, Inc., Lynchburg, Va., which include a local anesthetic. In addition to one or more anesthetics and protectants, pads for anorectal use include ingredients that provide a pleasant cooling sensation, such as eucalyptol and menthol. Customer satisfaction surveys establish that the cooling sensation provided by these ingredients is preferred for anorectal use.

Itching of the vulvar tissues may be especially uncomfortable. Furthermore, these tissues are quite sensitive in relation to rectal tissues and are easily irritated by products intended to relieve the itching discomfort associated with other tissue types. Conventional preparations for treating vulvar itching, such as Vagisil® cream, include benzocaine and are white in color. While an effective topical anesthetic, benzocaine can induce an undesirable sensitivity in some users with regular use. Furthermore, creams are messy and remain visible to the user after application. Other conventional preparations, such as the foams described in U.S. Pat. No. 6,818,204, are primarily for cleansing and only may include a small amount of protectant.

### SUMMARY

In one aspect, the invention provides an anti-itch composition that includes water, an anesthetic, from 5 to 45% of a protectant, a buffer, and a surfactant, where the composition excludes quantities of chemesthetic ingredients sufficient to provide a cooling effect when applied to vulvar tissues.

In another aspect of the invention, a method of forming an anti-itch cloth for treating itching of the vulvar tissues is disclosed. The cloth is wetted with an anti-itch composition including water, an anesthetic, from 5 to 45% of a protectant, a buffer, and a surfactant, where the composition excludes quantities of ingredients sufficient to provide a cooling effect when applied to vulvar tissues.

In yet another aspect of the invention, a method for treating vulvar discomfort is disclosed that includes contacting vulvar tissue with a cloth wetted with an anti-itch composition including water, an anesthetic, from 5 to 45% of a protectant, a buffer, and a surfactant, where the composition excludes quantities of chemesthetic ingredients sufficient to provide a cooling effect when applied to vulvar tissues.

### BRIEF DESCRIPTION OF THE DRAWINGS

The components in the drawings are not necessarily to scale, emphasis instead being placed upon illustrating the principles of the invention.

FIG. 1 represents anti-itch cloths for vulvar use enclosed by envelopes.

### DETAILED DESCRIPTION

While pads that effectively relieve the discomfort caused by anorectal itching are known, these pads are unsuitable to reduce the discomfort from itching of the vulvar tissues. Furthermore, these pads often are intended to be left in place to provide the maximum relief. The present invention provides disposable cloths that are designed to reduce itching discomfort when wiped across the vulvar tissue.

The cloths of the present invention include a fibrous substrate wetted with a composition that includes at least one anesthetic, protectant, buffer, and surfactant. The pH of the composition may be selected for compatibility with the vulvar tissues, thus reducing the possibility of irritation attributable to the composition. The composition also may be substantially clear, thus being nearly transparent to the user after application to the vulvar tissue. Thus, the present composition may be preferred by users when compared to conventional cream-based preparations that are opaque white.

Furthermore, the applicant has unexpectedly discovered that the cooling sensation that increases consumer acceptance for anorectal pads significantly reduces consumer acceptance for vulvar anti-itch cloths. In fact, when the ingredients that provide the cooling sensation for anorectal pads are utilized for vulvar use, individuals often describe an unpleasant burning or stinging sensation. Thus, the cloths of the present invention lack sufficient quantities of chemesthetic ingredients, such as menthol, eucalyptol, and the like, to provide the cooling sensation desired for anorectal pads. In one aspect, the present composition includes less than 0.018, 0.015, 0.013, or 0.008% chemesthetic ingredients.

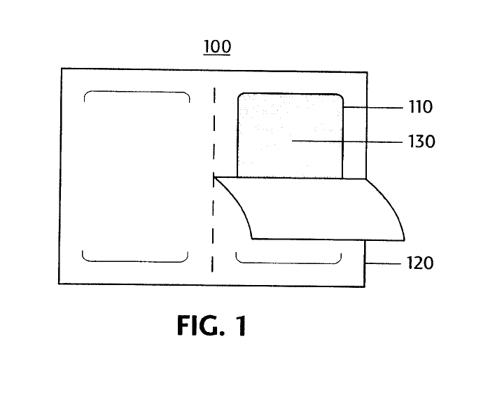

FIG. 1 represents two packaged anti-itch cloths 100. The package 100 includes an anti-itch cloth 110 for vulvar use in each of two envelopes 120. The cloth 110 is wetted with an anti-itch composition 130. While the term “cloth” is used for convenience, the material from which the cloth 110 is made may be any fibrous material capable of forming a web that is suitable for vulvar use and that is chemically compatible with the anti-itch composition 130. The cloth 110 may have a woven or non-woven structure.

The material from which the cloth 110 is made may include one or more natural or synthetic polymers, such as cotton, cellulose, nylon, rayon, and the like. In a preferred aspect, the cloth 110 is a non-woven spun lace, such as 55 g non-woven spun lace available from Ahlstrom-Green Bay, Green Bay, Wis. Non-woven spun lace may be preferred because it is soft, compatible with the anti-itch composition, and suitable for vulvar use. While the cloth 110 may be any size convenient for vulvar use, in one aspect, the cloth may be approximately 12.7 by 20.3 cm when unfolded.

The envelope 120 may be any material that encloses the cloth 110 and that substantially maintains the moisture content of the composition 130 prior to use. The envelope 120 may be foil, foil-lined paper, plastic, plastic-lined paper, combinations thereof, and the like. In one aspect, the envelope 120 may have an inner layer of plastic, a middle layer of paper, and an outer layer of foil. The plastic may be low density polyethylene (LDPE), linear low density polyethylene (LLDPE), combinations of these polymers, and the like.

While the envelope 120 may be any size convenient to enclose the cloth 110 in a folded or unfolded state, in one aspect, the envelope 120 is approximately 5.7 by 8.9 cm. The envelope 120 may be provided to the consumer in any convenient form, such as individually, or with multiple envelopes fully or partially attached at one or more edges.

The composition 130 serves to treat the discomfort caused by vulvar itching. This itching may result from abrasion of the tissues and/or from other causes including minor bacterial and/or fungal infection.

The anti-itch composition 130 may include one or more solvents, surfactants, buffers, and preservatives. In a preferred aspect, the ingredients of the composition are substantially clear when solvated or suspended in the solvent. By “substantially clear” it is meant that when the ingredients of the composition are combined, the composition has a turbidity at 24° C. of from 0.1 to 1 Nephelometric Turbidity Units (NTU) or from 0.3 to 0.8, when measured with a Micro100 turbidity instrument available from HF Scientific, Inc., Fort Myers, Fla. In a preferred aspect, a substantially clear composition has a turbidity from 0.45 to 0.65 NTU. Thus, the clarity of the composition may be within the range appropriate for municipal drinking water.

Preferably, the solvent includes water, with the composition preferably including from 70 to 95% or from 80 to 90% water, with about 82 to 87% water being more preferred. Unless stated otherwise, all percentages of ingredients in the composition are stated on a weight/weight (w/w) basis.

To assist in keeping the ingredients of the composition in solution, the composition may include a surfactant. Useful surfactants may be nonionic, cationic, anionic, or amphoteric in character. In one aspect, the surfactant may include octyl phenyl ethers, such as Octoxynol 9; alkylaryl polyether alcohols, such as TRITON®-X; alkyl poly glucosides, such as the PLANTAREN® surfactants available from Cognis Corp., Ambler, Pa.; or mixtures thereof.

In one aspect, the composition may include from 0.1 to 3% or from 0.1 to 1% of the surfactant. In a preferred aspect, the composition may include from 0.24 to 0.36% of the surfactant. In a more preferred aspect, the composition may include from 0.27 to 0.33% of the surfactant.

Unlike the anesthetic cleansing foam described in U.S. Pat. No. 6,818,204, which includes about 21% total surfactants to provide an effective cleanser, the anti-itch composition of the present invention uses a minor quantity of surfactant, preferably less than 10%, more preferably less than 5%, to enhance the clarity of the composition. Thus, the composition of the present invention lacks a sufficient amount of surfactants to serve as an effective cleanser.

The composition also may include a buffer to regulate the pH of the composition. The buffer may be selected to provide a pH to the composition that reduces the chance that irritation will be caused to the vulvar tissue through use of the anti-itch cloth. The buffer also may be selected to provide a pH that provides some inhibition to undesirable bacterial and/or fungal growth.

In a preferred aspect, the buffer is selected to provide a pH to the composition of from 3.4 to 4.1 (average of 3.75), which corresponds to the pH of the vulvar tissues. Due to the logarithmic nature of pH values, the average pH of the present composition is approximately an order of magnitude lower than the 4.0 to 5.5 (average of 4.75) pH value for FLEET® Anorectal Pads. In a more preferred aspect, the buffer is selected to provide a pH to the composition of from 3.4 to 3.9. In this manner, the pH of the composition is tailored to the lower pH value of the vulvar tissues as opposed to the higher pH of the rectal tissues. Thus, irritation to the vulvar tissues attributable to the use of the anti-itch cloth of the present invention may be reduced.

In one aspect, the buffer may include citric acid/sodium citrate, benzoic acid/sodium benzoate, acetic acid/sodium acetate, or mixtures thereof. At present, a more preferred buffer includes anhydrous citric acid and sodium citrate dihydrate. The composition may include from 0.1 to 1% or from 0.26 to 0.38% of anhydrous citric acid, in a preferred aspect, from 0.29 to 0.35%. The composition also may include from 0.1 to 1% or from 0.15 to 0.5% of the sodium citrate dihydrate, in a preferred aspect, from 0.2 to 0.27%.

While the anti-itch cloth 110 may be enclosed in the envelope 120 to maintain the effectiveness of the anti-itch composition 130, a preservative may be added to increase shelf-life. In one aspect, the preservative may include sodium benzoate, disodium edentate (EDTA), cetyl pyridinium chloride (CPC), benzalkonium chloride, or mixtures thereof. In a preferred aspect, the preservative includes sodium benzoate, disodium edentate, and cetyl pyridinium chloride in an approximately one to one to one mixture by weight. The composition may include from 0.1 to 3% or from 0.2 to 0.4% of the preservative, in a preferred aspect, from 0.27 to 0.35%.

Additionally, the composition includes one or more anesthetics and protectants as active ingredients. The anesthetic numbs the vulvar tissues, thereby reducing discomfort. In one aspect, the anesthetic may include pramoxine, benzocaine, dibucaine, tetracaine, resorcinol, or mixtures thereof. Preferable anesthetics form water-soluble salts that may be solubilized in water to provide substantially clear solutions. The concentration of anesthetic included in the composition may depend on the numbing ability of the anesthetic. For example, from 5 to 20% of benzocaine may be required to provide the same anesthetic effect as from 0.5 to 2% of pramoxine.

Pramoxine and its water-soluble salts may be preferred as an anesthetic because unlike some anesthetics, including benzocaine, pramoxine has a reduced tendency to sensitize the vulvar tissues. Pramoxine also may be preferred because when its water-soluble salt is solvated in water a substantially clear solution may be formed. The ability of pramoxine to form a substantially clear solution from its water-soluble salt directly contrasts with many anesthetics, including benzocaine, which forms a cloudy, opaque solution. In a more preferred aspect, the anesthetic includes from 0.5 to 2%, preferably from 0.8 to 1.5%, and more preferably from 0.9 to 1.1% of the hydrochloride salt of pramoxine.

By coating the vulvar tissue, the protectant may also provide a reduction in itching discomfort and protect the tissue from further irritation. In this manner, minor abrasions may be protected from further abrasion by the surrounding tissues and clothing. The abrasions also may be partially isolated from the atmosphere, thus assisting in keeping the tissue moist and reducing the likelihood fungal or bacterial attack. Preferable protectants are compatible with the other ingredients of the compositions and with vulvar use. More preferable protectants have the desired compatibility and also provide a substantially clear composition when mixed with the other ingredients.

Suitable protectants and preferred concentrations for protectant use in the present invention may be found in 21 C.F.R. §§310, 347, and 352 (Fed. Reg., Vol. 68, No. 107, Jun. 4, 2003, pp. 33362-81), for example. More preferred protectants and the weight percentage required for the protectants to qualify as a skin protectant under FDA guidelines are found in 21 C.F.R §247.10. Thus, more preferred protectants for use in the present composition include from 20 to 45% glycerin, from 1 to 30% dimethicone, or mixtures thereof. At present, glycerin is an especially preferred protectant. The composition may include from 5 to 45%, from 8 to 43%, preferably from 10 to 40%, and more preferably from 20 to 40% of the protectant.

In comparison to foaming compositions having a significant cleansing function, such as those described in U.S. Pat. No. 6,818,204, the present composition includes a greater protectant concentration. Furthermore, the increased protectant concentration of the present invention is incompatible with the formation of stable foams. Thus, the enhanced protectant concentration of the present anti-itch composition may provide better protection to the vulvar tissues in relation to foam-based cleaning products.

The composition 130 may be prepared by initially combining an acidic buffer ingredient, such as citric acid, with the highly water-soluble preservatives, such as cetyl pyridinium chloride and sodium benzoate, in water. To this stirred combination then may be added the protectant, basic buffer component, anesthetic, and disodium edentate. After continued stirring, the surfactant may be added. Additional ingredients, such as fragrances and the like that are compatible with vulvar tissues and the other ingredients of the composition also may be added. In one aspect, less than 1% of a fragrance may be added.

The cloth 110 may be wetted with the composition 130 by any convenient means, including soaking, spraying, brushing, and the like. In a preferred aspect, the cloth 110 may be placed in the envelope 120 to which from 4 to 5 g of the composition 130 may be added before the envelope 120 is sealed.

The following examples are provided to illustrate one or more preferred embodiments of the invention. Numerous variations can be made to the following examples that lie within the scope of the invention.

### EXAMPLES

### Example 1

An anti-itch composition including the ingredients and amount listed below in Table 1 was prepared.

TABLE 1

| Ingredient | Weight Percent (w/w) |
|---|---|
| Pramoxine HCl - USP | 1 |
| Glycerin 99% - USP | 12.5 |
| Octoxynol 9, NF Grade | 0.3 |
| Sodium Citrate (Dihydrate) - USP | 0.242 |
| Citric Acid (anhydrous) - USP | 0.322 |
| Sodium Benzoate - NF | 0.1 |
| Disodium Edetate - USP | 0.1 |
| Cetyl Pyridinium Chloride - USP | 0.1 |
| Water | 85.336 |

### Example 2

An anti-itch composition including the ingredients and amount listed below in Table 2 was prepared.

TABLE 2

| Ingredient | Weight Percent (w/w) |
|---|---|
| Pramoxine HCl - USP | 1 |
| Glycerin 99% - USP | 20 |
| TRITON-X | 0.3 |
| Sodium Citrate (Dihydrate) - USP | 0.24 |
| Citric Acid (anhydrous) - USP | 0.32 |
| Sodium Benzoate - NF | 0.1 |
| Disodium Edetate - USP | 0.1 |
| Cetyl Pyridinium Chloride - USP | 0.1 |
| Water | 77.84 |

### Example 3

An anti-itch composition including the ingredients and amount listed below in Table 3 was prepared.

TABLE 3

| Ingredient | Weight Percent (w/w) |
|---|---|
| Pramoxine HCl - USP | 1 |
| Glycerin 99% - USP | 20 |
| TRITON-X | 0.3 |
| Sodium Citrate (Dihydrate) - USP | 0.242 |
| Citric Acid (anhydrous) - USP | 0.322 |
| Sodium Benzoate - NF | 0.1 |
| Disodium Edetate - USP | 0.1 |
| Cetyl Pyridinium Chloride - USP | 0.1 |
| Plantaren 2000 | 0.005 |
| Fragrance | 0.02 |
| Water | 77.334 |

### Example 4

### Preparation of an Anti-Itch Composition

To 1621 kg of purified water was added 6.118 kg of citric acid (anhydrous) (granular) USP, 1.9 kg of cetyl pyridinium chloride USP, and 1.9 kg of sodium benzoate NF while stirring. Stirring of this mixture continued at room temperature for approximately 40 minutes after ingredient addition. Then, 237.5 kg of glycerin 99% USP, 4.588 kg of sodium citrate (dihydrate) USP, 19 kg of pramoxine HCl USP, and 1.9 kg of disodium edetate USP was added. Stirring of this mixture continued at room temperature for approximately 40 minutes after ingredient addition. Finally, 5.7 kg of Octoxynol 9, NF grade was added and the mixture was allowed to stir for an additional 40 minutes.

While various embodiments of the invention have been described, it will be apparent to those of ordinary skill in the art that other embodiments and implementations are possible within the scope of the invention. Accordingly, the invention is not to be restricted except in light of the attached claims and their equivalents.

1. An anti-itch cloth, for treating itching of the vulvar tissues, comprising:
a cloth wetted with a composition,
the composition comprising:
water,
an anesthetic,
from 5 to 45% of a protectant,
a buffer, and
a surfactant,
where the composition excludes quantities of chemesthetic ingredients sufficient to provide a cooling effect when applied to vulvar tissues.

2. The cloth of claim 1, where the composition has a turbidity of from 0.1 to 1 NTU.

3. The cloth of claim 1, where the composition has a turbidity of from 0.45 to 0.65 NTU.

4. The cloth of claim 1, where the composition has a pH of from 3.4 to 3.9.

5. The cloth of claim 1, where the composition has an average pH of about 3.7.

6. The cloth of claim 1, where the surfactant comprises less than 10% of the composition.

7. The cloth of claim 1, where the surfactant comprises less than 5% of the composition.

8. The cloth of claim 1, where the composition comprises at most 0.015% chemesthetic cooling ingredients.

9. The cloth of claim 1, where the anesthetic is selected from the group consisting of pramoxine, benzocaine, dibucaine, tetracaine, resorcinol, and mixtures thereof.

10. The cloth of claim 1, where the anesthetic comprises from 0.5 to 20% of the composition.

11. The cloth of claim 1, where the anesthetic comprises from 0.9 to 1.1% of the composition.

12. The cloth of claim 1, where the protectant is selected from the group consisting of glycerin, dimethicone, and mixtures thereof.

13. The cloth of claim 1, where the protectant comprises glycerin.

14. The cloth of claim 1, where the protectant comprises from 10 to 40% of the composition.

15. The cloth of claim 1, where the protectant comprises from 20 to 40% of the composition.

16. The cloth of claim 1, where the buffer is selected from the group consisting of citric acid/sodium citrate, benzoic acid/sodium benzoate, acetic acid/sodium acetate, and mixtures thereof.

17. The cloth of claim 1, where the buffer comprises from 0.1 to 1% of the composition.

18-20. (canceled)

21. A composition for treating vulvar tissue, consisting essentially of:
water,
an anesthetic,
from 5 to 45% of a protectant,
a preservative,
a buffer, and
a surfactant.

22-28. (canceled)

29. A method of forming an anti-itch cloth for treating itching of the vulvar tissues, comprising:
wetting a cloth with an anti-itch composition comprising:
water,
an anesthetic,
from 5 to 45% of a protectant,
a buffer, and
a surfactant, where the composition excludes quantities of ingredients sufficient to provide a cooling effect when applied to vulvar tissues.

30-40. (canceled)

41. A method for treating vulvar discomfort, comprising:
contacting vulvar tissue with a cloth wetted with an anti-itch composition comprising:
water,
an anesthetic,
from 5 to 45% of a protectant,
a buffer, and
a surfactant, where the composition excludes quantities of chemesthetic ingredients sufficient to provide a cooling effect when applied to vulvar tissues.

42-47. (canceled)

48. The anti-itch cloth of claim 1, where the surfactant is the sole surfactant.

49. The anti-itch cloth of claim 1, where the composition forms no stable foams.

50. The composition of claim 21, where the surfactant is the sole surfactant.

51. The composition of claim 21, where the composition forms no stable foams.

52. The method of claim 29, where the surfactant is the sole surfactant.

53. The method of claim 29, where the composition forms no stable foams.

54. The method of claim 41, where the surfactant is the sole surfactant.

55. The method of claim 41, where the composition forms no stable foams.

56. The anti-itch cloth of claim 1, where the composition comprises from 0.1 to 1% of the surfactant.

57. The anti-itch cloth of claim 1, where the composition comprises from 0.24 to 0.36% of the surfactant.

58. The anti-itch cloth of claim 1, where the composition comprises from 0.27 to 0.33% of the surfactant.
